(19) United States
(12) Patent Application Publication
(10) Pub. No.: US 2006/0242898 A1
(43) Pub. Date: Nov. 2, 2006

(54) Method of cultivating a biomass using the plant genus casuarina

(75) Inventor: Cliff Peiffer, Victoria Park (AU)

Correspondence Address: SMITH, GAMBRELL & RUSSELL, 1850 M STREET, N.W., SUITE 800, WASHINGTON, DC, 20036, US

(73) Assignee: BioEnergy Australia Pty. Ltd., Perth (AU)

(21) Appl. No.: 10/542,955

(22) PCT Filed: Jan. 22, 2003
(86) PCT No.: PCT/AU03/00065

§ 371 Date: Mar. 21, 2006

(51) Int. Cl. A01H 3/00; A01B 79/02

(52) U.S. Cl. 47/58.1 R

(57) ABSTRACT

The invention provides a method for of cultivating biomass, comprising the steps of: (a) Selecting at least a plant variety from the plant genus Casuarina capable of growing at a density equivalent to at least 5,000 stems per hectare for a period of 2 to 5 years; (b) Cultivating the plant variety for two to five years under suitable conditions to maintain the plantings at a density equivalent to at least 5,000 stems per hectare; and (c) Harvesting the resultant biomass between 2 and 5 years from the date of initial plantings.

### FIELD OF THE INVENTION

The present invention relates to methods for generating biomass. In particular, it provides methods of cultivating mixed species tree plantations capable of providing plant material for general biomass demands as well as hardwoods suitable for application in building, furniture etc.

### BACKGROUND ART

Due to the rapid increase in the world's population, there is a growing need for timber for building and furniture; woodchips for paper production; and, more recently, as an alternative renewable fuel source to replace fossil fuels. Demand for forest products is increasing, while large areas of forested land are being lost or degraded and timber harvest is being restricted in many of the world's natural forests.

Tree plantations, which are financially very attractive in many locations, offer the potential for meeting large portions of the world industrial wood needs. The supply of plantation-grown wood reduces the pressures and disturbances on old growth forests and natural timber stands. This is possible because the very high productivity of plantation forests requires less area to produce industrial wood. The use of plantations managed for timber production must increase to meet the world's increasing demand for wood and fibre from a reduced land base. Intensive management of plantation forests is perhaps the only way to meet the increasing demand for forest products and still reserve large areas of native forests for conservation and preservation purposes.

Many of the traditionally desirable plantation woods do not, however, grow fast enough to meet demands. Trees such as poplar (with its ‘short’ rotation period) require 20 years before they are mature enough for harvest. Hoop or exotic pines, which are often used for good-quality sawlogs or ply logs, take 25 to 45 years to mature, while eucalyptus sawlog plantations may require rotations of 40 years. Even eucalyptus wood for pulping may take 15 years for a rotation.

Despite the pressure to increase wood production from traditional timber plantations, it has been found that high density seedling planting has a negative impact on tree growth and development. Excessive tree density reduces individual tree diameter, which significantly impacts long-term plantation development. This growth loss can be expressed as reduced commercial wood yields, significantly higher harvesting costs and lower value products.

Initial planting densities typically range from 750 to 2500 stems/ha depending on management goals. Close spacing assists in the development of straight stems with fine branching but has a penalty in the development of stem size. Culling or thinning allows the best trees to develop but commonly thinnings may be cut for low-value products or may be wasted.

Plantation trees are typically planted as seedlings, having been grown for a year in a nursery. The cost of seedlings is much higher than that of seeds and planting out is labour intensive or requires specialised machinery.

During establishment of a seedling timber plantation and to a lesser extent over the life of the trees, the trees are extremely vulnerable to nutrient deficiencies as a result of poor soils and out-competition by weeds. Plantations thus require extensive weed control measures and regular application of suitable fertilisers. Problems are also encountered with pests and diseases, due to the vulnerable nature of monocultures. All of these factors can result in reduced growth and timber yield and poor quality timber.

In addition to the aforementioned problems, some highly desirable timber tree species, when grown in a plantation, require protection from the wind in order to grow and develop the tall, straight boles essential for the production of high-quality timber. One way around this problem is to first plant a ‘nurse’ plantation and then, a few years later, interplant with the desired timber species. This second species will then have the protection of the nurse trees and, just as importantly, the competition which will force it to grow tall and straight. After several years the nurse trees will be removed to allow full growth of the desired high value trees. This, however, is an expensive process and often economically unviable. Some desirable tree species have traits, such as allelopathic tendencies, which make them difficult to grow in closely spaced monoculture plantations.

Further, many high value timber species cannot be grown in specific regions and sites due to unfavourable environmental conditions such as rainfall, temperature, salinity, soil erosion or a high water table. This limits the range of sites that can be utilised for timber plantations and reduces the potential of timber plantations to reclaim sites such as degraded marginal agricultural land.

Thus, a range of problems have been encountered in meeting the growing demand for plantation timber, which cannot be met by traditional methods of planting and harvesting.

### SUMMARY OF THE INVENTION

In the work leading up to the present invention, the applicant has developed a novel system for generating large volumes of biomass of high quality and value in a minimum time frame and with limited plantation area. Typically, when plants are grown at high-density individual trees become suppressed and die because of the competition between the trees. Traditional plantation theories teaches that financial returns are maximised by planting stands at low tree stockings such that the trees are spaced at suitable intervals to minimise competition or by planting at high density but with substantial thinning in latter years to reduce competition between the trees. Further, traditional theory dictates that such plantings should be allowed to mature over at least 10 years to maximise biomass yields.

In one embodiment the invention provides a method for producing substantial quantities of biomass within a relatively short time period, comprising the steps of:
(a) Selecting at least a plant variety from the plant genus Casuarina capable of growing at a density equivalent to at least 5,000 stems per hectare for a period of 2 to 5 years;
(b) Cultivating the plant variety for two to five years under suitable conditions to maintain the plantings at a density equivalent to at least 5,000 stems per hectare; and
(c) Harvesting the resultant biomass between 2 and 5 years from the date of initial plantings.

Accordingly, in a second embodiment the invention provides a method for cultivating timber, comprising the steps of:
(a) Selecting at least a plant variety from the plant genus Casuarina capable of growing at a density equivalent to at least 5,000 stems per hectare for a period of 2 to 5 years;
(b) Cultivating the plant variety selected in step (a) under suitable conditions to maintain the plantings at a density equivalent to at least 5,000 stems per hectare; and
(c) Cultivating, in close proximity to the plant variety selected in step (a), at least a second plant species.

In a highly preferred form of the second embodiment there is provided a method of cultivating timber, comprising the steps of:
(a) Selecting at least a plant variety from the plant genus Casuarina capable of growing at a density equivalent to at least 5,000 stems per hectare for a period of 2 to 5 years;
(b) Cultivating for 2 to 5 years the plant variety selected in step (a) under suitable conditions to maintain the plantings at a density equivalent to at least 5,000 stems per hectare;
(c) Cultivating a second plant species capable of producing relatively high-value timber within about 10 to 15 years within approximately 0.5 to 5 meters of the plant variety cultivated in step (b); and
(d) Harvesting the plant variety cultivated in step (b) at repeat intervals of approximately 2 to 5 year until the second plant species has reached maturity or at least until it has reached a stage of harvest.

According to a third embodiment, the invention provides a method for producing Casuarina hybrid seeds comprising:

(a) Growing a first Casuarina species to sexual maturity and selecting plants of that species that have a phenotype of female fertility;
(b) Growing a second Casuarina species to sexual maturity and selecting plants of that species that have a phenotype of male fertility;
(c) Allowing cross-pollination between the female plants from step (a) with mature pollen from the male plants from step (b);
(d) Raising the female plants to produce hybrid seeds having genetic material from both parents; and
(e) Harvesting the hybrid seeds.

In a fourth embodiment the invention provides a method for raising Casuarina seedlings comprising the step of: cultivating the seedlings in the presence of a suitable water-absorbent paste or gel product.

Other aspects and advantages of the invention will become apparent to those skilled in the art from a review of the ensuing description, which proceeds with reference to the following illustrative drawings.

### BRIEF DESCRIPTION OF THE DRAWINGS

FIG. 1 illustrates the positions of Casuarina rows relative to plantings of high-value timber species Grevillea robusta and Toona ciliata. Note the spacings presented in the illustration are provided by way of example only. They are not intended to depict either the only way of growing these species or the most preferred way of achieving this outcome.

### DETAILED DESCRIPTION OF THE INVENTION

General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively, and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

As used herein the term “derived” and “derived from” shall be taken to indicate that a specific integer may be obtained from a particular source albeit not necessarily directly from that source.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the disclosure of the invention and apply throughout. Unless otherwise defined, all other scientific and technical terms used herein have the same meaning as commonly understood to one of ordinary skill in the art to which the invention belongs.

### DISCLOSURE OF THE PREFERRED EMBODIMENTS

The applicant has identified that plants within the genus Casuarina can be grown for a period of 2 to 5 years at high density with little or no detriment to the quality of the biomass generated. Such a result is significant as it provides a means to generate substantial amounts of biomass in a relatively short time period.

In one embodiment the invention provides a method for cultivating biomass, comprising the steps of:
(a) Selecting at least a plant variety from the plant genus Casuarina capable of growing at a density equivalent to at least 5,000 stems per hectare for a period of 2 to 5 years;
(b) Cultivating the plant variety for two to five years under suitable conditions to maintain the plantings at a density equivalent to at least 5,000 stems per hectare; and
(c) Harvesting the resultant biomass between 2 and 5 years from the date of initial plantings.

There are several advantages of growing Casuarina species. Those advantages include:
(i) They grow rapidly, providing shelter to other plant species grown in their proximity;
(ii) When Casuarina plants are grown in proximity to other species, the shelter they provide ensures the other species grow straight and tall;
(iii) They can be grown direct from seed;
(iv) They can be repeatedly coppiced and harvested; and
(v) They have the capacity to form symbiotic root nodules that are capable of fixing atmospheric nitrogen, which facilitates growth of other plants.

In addition to the above advantages, the calorific value of the component parts (eg leaves, branches, stems etc) of Casuarina plants is relatively uniform, unlike other plant varieties. Furthermore, the calorific value generated from Casuarina plants is believed to the highest or near the highest of any tree species. This feature of Casuarina plants is particularly advantageous where the biomass generated from the described method is employed for energy production. It also means that 100% of the harvested plant has a productive use, other than in the formation of mulch. In contrast, only about 70% of a forestry tree has a product use beyond serving as mulch.

The biomass productivity achieved from a planting of Casuarina plants will vary depending on, inter alia, the density of the planting. While Casuarina plants are grown in accordance with the broadest form of the invention at a density equivalent to at least 5,000 stems per hectare (i.e. 10,000 m²), it will be appreciated that the greater the density of the plantings, the higher the volume of biomass that may be recovered from the plantings. Thus, preferably the plants are grown at a density equivalent to at least 10,000 stems per hectare. More preferably, Casuarina plants are grown at a density equivalent to at least 20,000 stems per hectare. In a highly preferred form of the invention, Casuarina plants are grown at a density equivalent to between 40,000 and 60,000 (inclusive) stems per hectare.

As used herein “equivalent density” refers to a proportionate correlation of stem numbers based the size of the planting. Thus, if the area of planting is only half a hectare (i.e. 5000 m²) the proportionate number of stems grown in that field will be at least 2500. If the planting area is only one acre (4046.86 m²) the proportionate number of stems will be at least 2024. Conversely, if the planting is two hectares (ie 20,000 m²), the proportionate number of stems will be at least 10,000. Preferably, plantings will be arranged in approximately 1 acre to approximately 1 hectare sized plots. More, preferably the plantings will be prepared in approximately 1 hectare sized plots.

The plantings described herein may be laid out as a single bed of plants or in multiple beds covering a selected field size. Preferably the Casuarina plants are grown in multiple planting beds each of which is of a width and length that facilitates easy access for maintenance of the plants and for harvesting. Where the Casuarina plants are grown in hectare sized plots a plurality of planting beds will preferably be used in the planting regime.

Where Casuarina plants are grown at a density equivalent to at least 5,000 stems per hectare the plantings are harvested at relatively regular intervals. Harvesting times of plantings will vary depending on the particular Casuarina variety used in the method as well as factors such as the rate at which the variety matures the environment in which it is grown and the amount of biomass generated per plant. Preferably, the plantings are harvested within 2 to 5 years from planting. More preferably, they are harvested within 2 to 4 years, while harvesting at 3-years from planting is highly preferred.

Biomass productivity levels for any particular planting will vary depending on, inter alia, the Casuarina variety planted, the health of the plants over the growing period, the number of stems grown and the age of the trees. Where Casuarina plantings are prepared in hectare plots, plantings of 5000 stems per hectare and harvesting after approximately 3 years of growth will lead to approximately 35 tonnes of biomass per hectare. Planting densities around 10,000 stems per hectare with harvesting after approximately 3 years of growth will lead to biomass productivity levels of approximately 60 tonnes per hectare. While planting densities at around 20,000 stems per hectare with harvesting after approximately 3 years of growth will lead to biomass productivity levels of approximately 100 tonnes per hectare. As 100% of the harvested plant has productive value, the biomass productivity levels identified herein are based on the total volume of harvested material. While these productivity levels are based on plantings prepared in hectare plots the invention is not so limited in its scope to plantings in hectare plots.

Generally, harvesting will involve cutting individual Casuarina stems adjacent to, but above, the roots of the variety. When cut in this manner Casuarina plants will re-shoot from the root system left in the ground. Thus, where at least the root system is retained in the ground a plantation may be re-established from the existing roots. Cultivation of Casuarina plants in this manner permits repeat harvesting of the plantings. Preferably, the plantings are harvested every 2 to 5 years from planting or the last harvest. More preferably they are harvested every 2 to 4 years, while harvesting at 3-years intervals is highly preferred.

Typically, where a 3-year growth and harvest regime is used, this regime may be repeated between 1 and 6 times using the same rootstock. Most desirably, the growth and harvest regime is repeated 1 to 4 times and in one example of the invention the growth and harvest regime is repeated 3 times.

Where Casuarina plants are harvested at repeated intervals, the sustainability of plant growth between the harvest times will depend on the type of soil in which the plants are cultivated and the nutrients and minerals in the soil, as well as a range of other silviculture limiting factors such as whether any disease affects the rootstock through poor cutting techniques, the inability of the plants to sustain regular harvesting, and seasonal impacts on the production of shoots. Those skilled in the horticultural field will recognise that many of these factors can be controlled through the use of fertilisers and minerals, where appropriate, as well as through careful silviculture techniques.

Methods for selecting Casuarina plant varieties for use in the present invention will be known to those experienced in the horticultural field. As an example only, the selected species may be grown at a desired density in test plots and examined for detrimental effects to plant growth. Preferably, the plant variety is selected from the group comprising: Casuarina cunninghamiana, Casuarina glauca or Casuarina obesa or a hybrid developed from these varieties.

In a preferred form of the invention, the plant variety selected for use in the method is a hybrid generated by crossing Casuarina cunninghamiana, Casuarina glauca or Casuarina obesa with one of the other aforementioned species. Most preferably, the hybrid variety is generated by crossing Casuarina cunninghamiana and Casuarina glauca. Hybrids between Casuarina cunninghamiana and Casuarina glauca have many desirable attributes, some of which include: the hybrid produces very straight, tall stems on a wide range of sites including those where rainfall is moderate and access to groundwater is limited. The hybrid has the potential to be grown in more saline situations than Casuarina cunninghamiana and, from preliminary observations of growth, is able to grow more vigorously on a wider range of sites. It exhibits a high degree of salt tolerance, is suitable as biofuel, exhibits nitrogen fixing capacity, can be direct seeded and coppiced.

The methods described herein are not limited to the growth of a single Casuarina species within the stem densities described. Rather a plurality of different Casuarina species may be grown in a single stand. Where more than one Casuarina species is grown in a single stand the combined total of plant species should amount to the stem densities described herein.

In addition to identifying the above-mentioned benefits of Casuarina plants, the inventors have discovered that the combined protection provided by the Casuarina plantings and their nitrogen fixing capacity generates a unique environment in which to grow plant varieties of a second species. Accordingly, in a second embodiment the invention provides a method of cultivating timber, comprising the steps of:
(a) Selecting at least a plant variety from the plant genus Casuarina capable of growing at a density equivalent to at least 5,000 stems per hectare for a period of 2 to 5 years;
(b) Cultivating the plant variety selected in step (a) under suitable conditions to maintain the plantings at a density equivalent to at least 5,000 stems per hectare; and
(c) Cultivating, in close proximity to the plant variety selected in step (a), at least a second plant species.

The ability of Casuarina plants to grow rapidly and at densities as herein described provides a significant advantage to the growth of the second plant species grown in their proximity. The shelter provided by the Casuarina plants promotes the second plant species growing straight and tall. While any plant species may be selected to capitalise on this attribute preferably the second plant species is a high value timber generating species that takes approximately 8 to 20 years to reach maturity. More preferably the second species will reach maturity within 9 to 18 years and even more preferably within about 10 to 15 years.

As used herein “high value timber species” refers to any plant species that produces a timber product that has commercial value. A person of ordinary skill in the field of horticulture will recognise that a wide range of trees produce high value timber, any of which may be employed in the present invention. In a preferred form of the invention the “high value timber” producing trees are selected from the varieties: Grevillea robusta (silky oak) or Toona ciliata (red cedar).

In order to avoid damages to the second plant species grown during harvesting of the Casuarina plants the second plant species will preferably be grown proximate to but on the periphery of the Casuarina plantings.

In order to maximise the protective and nutrient benefits obtainable from the Casuarina plantings, the second species should be grown within 0.5 to 5 meters of the Casuarina plantings. Such distances will vary depending on the type of plant species selected as the second species. Preferably, where the second species grown is a high value timber species, trees of the species will be grown within 1 to 3 meters from the Casuarina plantings. More preferably, the second species is grown within 1 to 2 meters from the Casuarina plantings, with an example of the distance between the second species and the Casuarina plantings being 1.3 to 1.5 meters.

To avoid competition between plants of the second species, individual plants should also be spaced at suitable distances to avoid such deleterious effects. Such distances will vary depending on the type of plant species selected as the second species. Preferably, where the second species grown is a high value timber species, trees of the species will be grown at distances of approximately 1 to 5 meters apart. More preferably, the second species is grown between 2 to 4 meters apart, with an example of the distance between trees of the second species being about 3 meters apart.

Where the second plant species takes more than approximately 3 years to reach maturity, preferably the Casuarina plants are harvested at repeat intervals as described above, until the second species reaches maturity or at least until it is ready to be harvested.

In a highly preferred form of the second embodiment of the invention there is provided a method of cultivating timber, comprising the steps of:
(a) Selecting at least a plant variety from the plant genus Casuarina capable of growing at a density equivalent to at least 5,000 stems per hectare for a period of 2 to 5 years;
(b) Cultivating for 2 to 5 years the plant variety selected in step (a) under suitable conditions to maintain the plantings at a density equivalent to at least 5,000 stems per hectare;
(c) Cultivating a second plant species capable of producing relatively high-value timber within about 10 to 15 years within approximately 0.5 to 5 meters of the plant variety cultivated in step (b); and
(d) Harvesting the plant variety cultivated in step (b) at repeat intervals of approximately 2 to 5 year until the second plant species has reached maturity or at least until it has reached a stage of harvest.

The subject method is not limited to the growth of a single high-value timber species, but includes the growth of a plurality of any number of these species. Where a plurality of high-value timber species are grown in proximity to Casuarina plantings the ratio of the different timber species may be one to one or any other desirable ratio. Such ratios will be determined by a host of factors including, inter alia, the timber species selected and the particular uses to which the timber will be placed.

According to a highly preferred form of the invention, both Grevillea robusta and Toona ciliata are grown in close proximity to the Casuarina plantings. When grown together, both Grevillea robusta and Toona ciliata may be grown in any ratio. Preferably, however, approximately 50 to 95 percent of the crop will be Grevillea robusta, while about 5 to 50 percent will be Toona ciliata. An example of a ratio that is detailed herein is 80% Grevillea robusta, to 20 percent Toona ciliata.

Where more than one high-value timber species is grown, the species may be grown in any planting pattern or combination of patterns suitable for commercial production of the plants. Such planting patterns will be known to those skilled in the growth of such timber. Preferably, the high-valued species are widely spaced to encourage diameter growth and avoid the problems of allelopathy (i.e. inability of the species to grow in close proximity). This may be achieved, for example, by spacing the plants in alternating rows.

Irrespective of the planting pattern chosen, the high value timber species should be planted in such a fashion to capture the shelter provided by the Casuarina plantings. This might be achieved, for example, by growing the high-value timber species between rows or sets of Casuarina plantings.

Pruning, harvesting, and/or thinning of the high value timber species should be carried out at regular intervals to maximise the size and quality of the timber produced. In this respect, removal of branches at various heights during the growth of a tree will allow most trees to develop knot free wood with minimal stem taper.

Repeat harvesting and coppicing the Casuarina plants every two to five years will keep the Casuarina plants from competing with the high value timber species. A person skilled in the art will be able to determine a pruning, harvesting, coppicing and thinning timetable, which will produce trees of a desired size and quality.

In addition to developing the above method the inventors have also developed a functional method for producing Casuarina hybrids. Unlike most plant varieties plants within the species Casuarina are dioecious. Accordingly, to produce Casuarina hybrids from seed, special seed orchards are required to generate the seed. According to a third embodiment, the invention provides a method for producing Casuarina hybrid seeds comprising:
(a) Growing a first Casuarina species to sexual maturity and selecting plants of that species that have a phenotype of female fertility;
(b) Growing a second Casuarina species to sexual maturity and selecting plants of that species that have a phenotype of male fertility;
(c) Allowing cross-pollination between the female plants from step (a) with mature pollen from the male plants from step (b);
(d) Raising the female plants to produce hybrid seeds having genetic material from both parents; and
(e) Harvesting the hybrid seeds.

According to this method the first and second species are preferably grown in the same orchard in alternate rows. When the plants of the first Casuarina species near sexual maturity, all the males of the species are culled from the orchard. Likewise when the second Casuarina species near sexual maturity, all the females of the species are culled from the orchard. Following culling remaining male Casuarina species are allowed to pollinate the remaining female Casuarina species. This may be achieved either naturally or using any means known in the field. Provided the orchard is sufficiently isolated, seed produced according to this method should largely be hybrid seed. In one example of the present invention the female Casuarina species will be C. glauca while the male Casuarina species will be C. cunninghamiana.

In a further refinement of the described method, male and female Casuarina species exhibiting desired attributes may also be selected during culling of the alternate sex plant species. By simply selecting the best individuals within a family and ultimately by eliminating poorly performing families, superior quality Casuarina hybrid species can be selected for.

Within the seed trade industry, hybrid seeds command a pre-eminent position because of their superior vigour, uniformity and performance. Accordingly, the present invention also resides in Casuarina hybrid seed produced according to this method. From hybrid seed, Casuarina plants may be generated.

Casuarina hybrids produced according to the present invention may be manipulated to enhance their phenotypic or genotypic characteristics. Such techniques for manipulating the hybrids will be known to those of skill in plant breeding and will include cloning, genetic recombination, or further breeding. Thus, the progeny of the initial cross may follow any one of the paths outlined below:
1) It may be crossed with either of its parents. Such back crossing would have the effect of enriching the characteristics of that parent in the next generation.
2) It may be crossed with a plant of the same species as one of its parents, either to introduce new characters or to affirm certain characteristics of that species.
3) It may be crossed with a plant from a species different from those of either of its parents. Such would have the effect of introducing further new characteristics.
4) It may be crossed with a plant species that is itself the product of one or more crosses.

Such breeding programmes are cumulative and will span a number of years, with careful selection taking place at each stage of the programme. By engaging in such programmes new and unique Casuarina plant varieties may be generated.

Casuarina plants may be propagated by either direct-seeding into the field or transplanting small seedlings into the ground. Seedlings are separated, dipped into a suitable water-absorbent paste or gel product, and planted directly into the field using appropriate machinery. This method dramatically reduces establishment risk and makes subsequent control of weeds simpler.

According to a fourth embodiment the invention provides a method for raising Casuarina seedlings comprising the step of: cultivating the seedlings in the presence of a suitable water-absorbent paste or gel product.

In one form of the invention Casuarina seed and seedlings are cultivated in the presence of at least a wetted water-absorbent polymers. In general, suitable water-absorbent polymers consist of one or more granular materials that, when wetted, convert to a gel or paste form and are capable of absorbing and releasing water repeatedly. Suitable polymers will absorb up to several hundred times their weight in water and can repeatedly absorb and release the water for as long as a decade. When placed near the roots of Casuarina plants, such polymers act as “water banks,” absorbing excess water until the Casuarina plant roots can tap it. When properly applied, suitable polymers can reduce the amount of water lost through percolation and evaporation, reduce the leaching of soil nutrients, pesticides, or herbicides, and improve soil aeration. Those effects improve yields and reduce watering cost significantly.

Certain water-absorbent polymers are known to speed the growth of commercially valuable plants through improved water management. Polymers that may be used for this purpose include polyacrylate (the absorbent agent used in disposable diapers), polyvinyl alcohol, starch-based copolymers, and cross-linked polyacrylamides. More specific examples of water-absorbent polymer mixes that might be used include, aqueous gels derived from highly absorbent, cross-linked, mixed salts of homopolymerised or copolymerised acrylic acid; synthetic agricultural polymers such as polyelectrolytes used in combination with water-soluble polysaccharides; dissolved water-soluble polyisocyanete capped prepolymers; substantially non-ionic polyacrylamides cross-linked with a low amount of methylenebisacrylamide (MBE); or hydro-gel polymers derived from polyvinyl alcohol and polyacrylic acid. Blends of such agents and compositions that are commercially available may also be used for this purpose.

If desired, such polymers can also be mixed with soil, nutrient supplement or any other agricultural mixture (eg synthetic growth media) that aids the growth of seedlings.

Biomass harvested from Casuarina plants has a wide range of potential uses. One particular use is as a biofuel. Casuarina wood is easy to split, has a high calorific value (around 5000 kcal/kg) and burns slowly with little ash. Converting this to MJ/kg (1 kcal/kg=4.1868 kJ/kg) the calorific value is 20.934 MJ/kg. The energy embodied in wood is largely a function of mass and moisture content. A typical figure used is 20 MJ/kg (IGPO 2001) for dry wood. As the calorific value of Casuarina plants is relatively uniform across the component part of the plant, preferably the biofuel is prepared from the leaves, branches and those parts of the stem that are not capable of alternate uses such as those mentioned below.

An alternate use for harvested biomass is in the preparation of composite boards, which may be prepared according to standard technology well known in the field. Most preferably, the composite boards are prepared from a hybrid of Casuarina cunninghamiana and Casuarina glauca plants. Analysis of composite boards produced from such hybrids has revealed that this Casuarina hybrid wood species produces composite boards typical of most hardwoods and very similar to eucalyptus, and is an acceptable fibre furnish for composite board production if the fibre preparation process is correctly adjusted to suit hardwood short fibre. In comparison to 100% Pinus radiata, the strength properties of 100% Casuarina composite boards are typical of hardwood composite boards. However, when blended with Pine, the board properties improve substantially. The outstanding property of this Casuarina hybrid fibre is its light colour, which compliments the light colour of pine fibre and produces a visually attractive board product.

Without being limited in any context as to the possible uses of the biomass produced by the method of the invention, the biomass produced has been found to be particularly useful in the preparation of chicken litter, charcoal products and for the preparation of pulp in addition to its uses as a biofuel and in the preparation of composite boards.

### BEST MODE(S) FOR CARRYING OUT THE INVENTION

The features of the present invention are more fully described in the following non-limiting Examples. It is to be understood that these examples are included solely for the purposes of exemplifying the various embodiments of present invention. They should not be understood, in any way, as a restriction on the broad description of the invention as set out above.

### EXAMPLE 1

### Positions of Plant Species

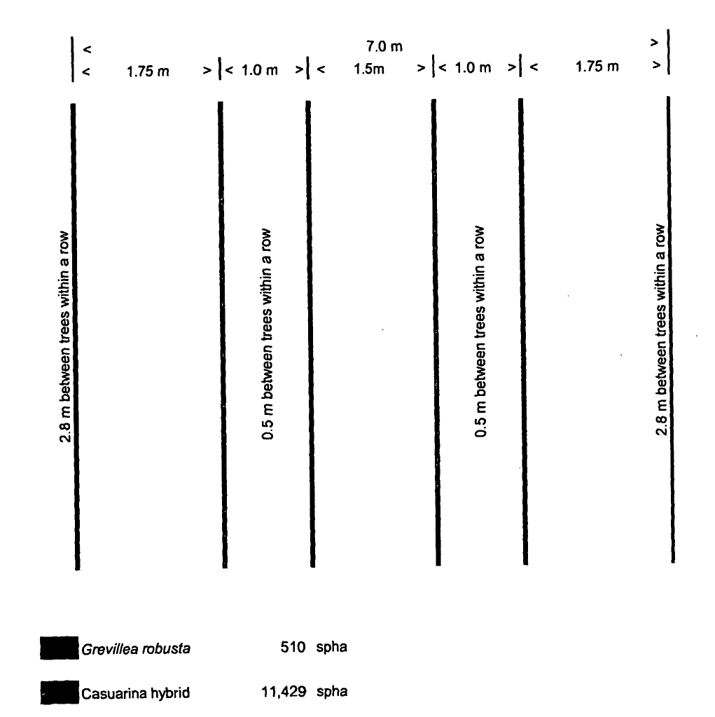

Hybrids between Casuarina cunninghamiana and Casuarina glauca were planted at approximately 20,000 stems/hectare in 2 beds next to one another, in 1.0 metre wide biomass beds in accordance with FIG. 1. The distance between each biomass bed was approximately 1.5 metres and at the head of each row there was a headrow. Within each of the biomass beds there are two biomass rows, 1.0 m apart with the outside row being 1.75 m from the edge of the bed.

Adjacent to each of the biomass beds high-value timber species Grevillea robusta and Toona ciliata, were grown. These varieties were planted at a density of 450 stems/hectare and 50 stems/hectare respectively, at approximately 1.75 metres from the beds in accordance with FIG. 1 (see also Table 1).

After approximately 3 years, the Casuarina plants are harvested by cutting the stems near ground level adjacent to the roots. Rootstock left in the ground is allowed to re-shoot, providing a means for re-establishing the Casuarina plant rows. Harvested biomass may be used for a range of different purposes including but not limited to the preparation of composite boards, as a biofuel, for charcoal production etc.

After approximately 3 years the Grevillea robusta and Toona ciliata species should be thinned to reduce their numbers to 225 and 25 stems/hectare, respectively.

Pruning of the Grevillea robusta and Toona ciliata branches is again carried out after approximately 6.5 years in accordance with Table 1. Briefly, both Grevillea robusta and Toona ciliata are pruned at various heights to allow the trees to grow tall and straight. Further, harvesting and thinning of the Grevillea robusta and Toona ciliata is carried out as necessary to reduce competition between the plants.

After a further 3 years (year 6) the Casuarina plants are again harvested according to the previous method. Rootstock is also allowed to re-shoot.

In years 7, 8 and 10 the Grevillea robusta and Toona ciliata are again pruned in accordance with Table 1 to allow the trees to continue grow tall and straight. Final harvesting of the Casuarina plants is undertaken at year 10 in accordance with Table 1. At the same time both the Grevillea robusta and Toona ciliata plantings should be thinned to allow the remaining trees to achieve maximum diameter and wood quality prior to harvesting at approximately year 15.

Table 1 provides a pruning, harvesting, coppicing and thinning regime

| | | | Hybrid |
|---|---|---|---|
| | | | between |
| | | | C. |
| | Grevillea | Toona | cunninghamiana |
| Action | robusta | ciliata | and C. glauca |
| Plants per hectare | 450 | 50 | 20 000 |
| Age 3 Harvest Hybrid & coppice | | | 20 000 |
| Age 3 Prune 250/hectare | 225 | 25 | |
| to 2.2 m | | | |
| Age 6.5 Prune 250/hectare | 225 | 25 | |
| to 4.4 m | | | |
| Age 6.5 Prune 50/hectare to | 225 | 25 | |
| 6.5 m | | | |
| Age 6.5 Thin to waste | 225 | 25 | |
| Age 7 Harvest hybrid and | | | 20 000 |
| coppice | | | |
| Age 7.2 Prune 125/hectare to | 100 | 25 | |
| 8.2 m | | | |
| Age 8.1 Prune 125/hectare | 100 | 25 | |
| 10.0 m | | | |
| Age 10 Thin 125/hectare | 100 | 25 | |
| Age 10 Harvest hybrid and | | | 20 000 |
| coppice | | | |
| Clearfall harvest 125/hectare | 100 | 25 | |

### EXAMPLE 2

This Example illustrates a particular use to which biomass of the present invention may be put. Other uses will be recognised by those skilled in the art. In the following particular Example Casuarina biomass was used to prepare composite boards in a 100% Casuarina biomass preparation and in a mixed preparation.

Freshly harvested Casuarina and Pine logs were manually debarked and then chipped in a 50 hp, 36″ Bruks chipper. The casuarinas bark was difficult to remove as it is more strongly bonded and of a stringy nature. The Casuarina logs were estimated to be in the 3-5 year age range and the Pinus radiata in the 9-12 year range. The chips were vibratory screened to remove over and undersize pieces.

The Pinus radiata logs were processed separately and mixed with casuarinas to evaluate the suitability of a blend of these two materials. After the logs were debarked, chipped and screened, fibers were produced with separate runs for casuarinas and Pinus radiata, followed by a mixed chip run with pine and Casuarina chips in the mix ratio 60:40. In all runs the chips were pre-steamed in a feed bin, fed by high compression screw feeder to a pressurized heating vessel and through the Sunds CD300 Defibrator from which the fibers were blown to a cyclone at atmospheric pressure. The first run on the Casuarina was repeated after recalibration of the feed rate. The pine was heated to 170° C. for 180 seconds, the Casuarina heated to 165° C. for 120 seconds, and the Pine:Casuarina mix heated to 175° C. for 120 seconds. In all runs, the Sunds 3847 unidirectional plate pattern was used and the Defibrator plate gap adjusted between 0.05 mm and 0.10 mm depending on the fibre.

Each batch of fibre from the separate runs was air dried in a static drying cupboard over a 24 hour period to approx 3% moisture content.

Subsequently, individual batches of fibre weighing 12 kg were air gun sprayed with melamine urea formaldehyde resin and mixed in a mechanical blender with continuous pneumatic circulation to ensure maximum individual fibre to resin contact. Wesfi Ltd supplied the melamine urea formaldehyde resin used with a solids concentration of 63% and the addition to fibre was 14% on a dry resin to fibre solids ratio. The wax used, also supplied by Wesfi Ltd, was an emulsified petroleum based wax of solids content 60%, mixed with the resin solution at 0.6% dry wax/fibre solids ratio.

The resinated fibers were subsequently pressed to form board samples. Individual fibre mats of area dimension 300×300 mm were manually formed. The mats were placed in a heated hydraulic press and pressed for approximately 210 seconds at 200° C. to obtain the required thickness and density.

Results show that this Casuarina hybrid wood species produces fibre typical of most hardwoods and very similar to eucalyptus, and is an acceptable fibre fumish for composite board production if the fibre preparation process is correctly adjusted to suit hardwood short fibre. In comparison to 100% Pinus radiata, the strength properties of 100% Casuarina composite boards are typical of hardwood composite boards. However, when blended with Pine, the board properties improve substantially. The outstanding property of this Casuarina hybrid fibre, is its light colour, which compliments the light colour of Pine fibre and produces a visually attractive board product.

1. A method for cultivating biomass, comprising the steps of:
(a) Selecting at least a plant variety from the plant genus Casuarina capable of growing at a density equivalent to at least 5,000 stems per hectare for a period of 2 to 5 years;
(b) Cultivating the plant variety for two to five years under suitable conditions to maintain the plantings at a density equivalent to at least 5,000 stems per hectare; and
(c) Harvesting the resultant biomass between 2 and 5 years from the date of initial plantings.

2. The method according to claim 1, wherein Casuarina plants are grown at a density equivalent to at least 10,000 stems per hectare.

3. The method according to claim 1, wherein Casuarina plants are grown at a density equivalent to at least 20,000 stems per hectare.

4. The method according to claim 1, wherein Casuarina plants are grown at a density equivalent to between 40,000 and 60,000 (inclusive) stems per hectare.

5. The method according to claim 1, wherein Casuarina plants are grown at a density equivalent to approximately 42,000 stems per hectare.

6. The method according to claim 1, wherein the Casuarina plantings are harvested within 2 to 4 years

7. The method according to claim 1, wherein the Casuarina plantings are harvested at 3-years from planting.

8. The method according to claim 1, wherein during harvesting individual Casuarina stems are cut adjacent to, but above, the roots of the variety.

9. The method according to claim 8, wherein re-growth of the Casuarina plants is fostered following harvesting of the plantings.

10. The method according to claim 9, wherein the plantings are harvested every 2 to 5 years from planting or the last harvest.

11. The method according to claim 9, wherein the plantings are harvested every 2 to 4 years.

12. The method according to claim 9, wherein the plantings are harvested at 3-years intervals.

13. The method according to claim 1, wherein the Casuarina plant variety is selected from the group comprising: Casuarina cunninghamiana, Casuarina glauca or Casuarina obesa or a hybrid developed from these varieties.

14. The method according to claim 13, wherein the Casuarina plant variety is a hybrid generated by crossing Casuarina cunninghamiana, Casuarina glauca or Casuarina obesa with one of the other aforementioned species.

15. The method according to claim 13, wherein the Casuarina plant variety is a hybrid variety generated by crossing Casuarina cunninghamiana and Casuarina glauca.

16. A method for cultivating timber, comprising the steps of:
(a) Selecting at least a plant variety from the plant genus Casuarina capable of growing at a density equivalent to at least 5,000 stems per hectare for a period of 2 to 5 years;
(b) Cultivating the plant variety selected in step (a) under suitable conditions to maintain the plantings at a density equivalent to at least 5,000 stems per hectare; and
(c) Cultivating, in close proximity to the plant variety selected in step (a), at least a second plant species.

17. The method according to claim 16, wherein the second plant species takes approximately 3 to 20 years to reach maturity.

18. The method according to claim 16, wherein the second plant species takes between 6 to 18 years to reach maturity.

19. The method according to claim 16, wherein the second plant species takes between 10 to 15 years to reach maturity.

20. The method according to claim 16, wherein the second plant species is high value timber species.

21. The method according to claim 16, wherein the second plant species is selected from the varieties: Grevillea robusta (silky oak) or Toona ciliata (red cedar).

22. A method for cultivating timber, comprising the steps of:
(a) Selecting at least a plant variety from the plant genus Casuarina capable of growing at a density equivalent to at least 5,000 stems per hectare for a period of 2 to 5 years;
(b) Cultivating for 2 to 5 years the plant variety selected in step (a) under suitable conditions to maintain the plantings at a density equivalent to at least 5,000 stems per hectare;
(c) Cultivating at least a second plant species capable of producing relatively high-value timber within about 10 to 15 years within approximately 0.5 to 5 meters of the plant variety cultivated in step (b); and
(d) Harvesting the plant variety cultivated in step (b) at repeat intervals of approximately 2 to 5 year until the second plant species has reached maturity or at least until it has reached a stage of harvest.

23. The method according to claim 22, wherein a plurality of high-value timber species are grown in proximity to Casuarina plantings.

24. The method according to claim 23, wherein both Grevillea robusta and Toona ciliata are grown in close proximity to the Casuarina plantings.

25. The method according to claim 22, wherein the second plant species selected in step (c) is harvest between 10 and 15 years from initial planting.

26. A method for producing Casuarina hybrid seeds comprising:
(a) Growing a first Casuarina species to sexual maturity and selecting plants of that species that have a phenotype of female fertility;
(b) Growing a second Casuarina species to sexual maturity and selecting plants of that species that have a phenotype of male fertility;
(c) Allowing cross-pollination between the female plants from step (a) with mature pollen from the male plants from step (b);
(d) Raising the female plants to produce hybrid seeds having genetic material from both parents; and
(e) Harvesting the hybrid seeds.

27. A method for raising Casuarina seedlings comprising the step of: cultivating the seedlings in the presence of a suitable water-absorbent paste or gel product.
